(19) United States
(12) Patent Application Publication
(10) Pub. No.: US 2023/0357172 A1
(43) Pub. Date: Nov. 9, 2023

(54) Perfluorinated Allyl Ethers and Perfluorinated Allyl Amines and Methods of Making and Using the Same

(71) Applicant: 3M INNOVATIVE PROPERTIES COMPANY, St. Paul, MN (US)

(72) Inventors: Sean M. Smith, Woodbury, MN (US); Klaus Hintzer, Kastl (DE); Markus E. Hirschberg, Mühldorf (DE); William M. Lamanna, Stillwater, MN (US)

(21) Appl. No.: 17/911,567

(22) PCT Filed: Feb. 25, 2021
(86) PCT No.: PCT/IB2021/051578

§ 371 Date: Sep. 14, 2022

Related U.S. Application Data
(60) Provisional application No. 62/991,636, filed on Mar. 19, 2020.

(51) Int. Cl. C07D 207/16; C07C 211/24; C07C 43/192; C07D 241/06; C07D 223/04; C07C 49/167; C07C 49/395; C07C 49/403; C07D 265/30; C07D 221/00; C07C 43/17; C07D 207/22

(52) U.S. Cl. CPC C07D 265/30; C07C 43/17; C07C 43/192; C07C 49/167; C07C 49/395; C07C 49/403; C07C 211/24; C07D 207/16; C07D 207/22; C07D 221/00; C07D 223/04; C07D 241/06

(57) ABSTRACT

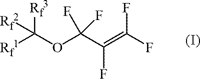

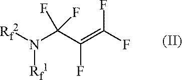

Described herein is a method to synthesize a perfluorinated allyl ether compound of formula (I) or a perfluorinated allyl amine compound of formula (II) Where R_{f}¹ and R_{f}² are (i) independently selected from a perfluorinated alkyl group comprising 1-7 carbon atoms, a perfluorinated aryl group comprising a 5- or 6-membered ring, or combinations thereof, and optionally comprising one or more catenated heteroatoms selected from N or O; or (ii) bonded together to form a perfluorinated ring structure having 4-8 ring carbon atoms, optionally comprising at least one catenated O or N atom; and R_{f}³ is a perfluorinated alkyl group comprising 1-3 carbon atoms. The resulting perfluorinated allyl ether compounds disclosed herein may be used in polymer synthesis.

### TECHNICAL FIELD

The present disclosure relates to perfluorinated allyl ethers and perfluorinated allyl amines and methods of making such compounds and using the perfluorinated allyl ethers in the synthesis of fluoropolymers.

### SUMMARY

There continues to be a need for novel perfluorinated monomers and fluoropolymers derived therefrom.

In one aspect, a perfluorinated allyl ether compound is provided. The perfluorinated allyl ether compound is represented by the following general formula (I):

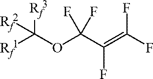

Where R_{f}¹ and R_{f}² are (i) independently selected from a perfluorinated alkyl group comprising 1-7 carbon atoms, a perfluorinated aryl group comprising a 5- or 6-membered ring, or combinations thereof, and optionally comprising one or more catenated heteroatoms selected from N or O; or (ii) bonded together to form a perfluorinated ring structure having 4-8 ring carbon atoms, optionally comprising at least one catenated O or N atom; and
R_{f}³ is a perfluorinated alkyl group comprising 1-3 carbon atoms.

In one aspect, a method for making a perfluorinated allyl ether is disclosed. The method comprising: contacting a perfluoroketone or perfluorinated acid fluoride with a metal or ammonium perfluorocarbanion salt in an aprotic solvent to form a perfluorinated tert-alkoxide salt; and

contacting the perfluorinated tert-alkoxide salt with a perfluoroallylating reagent to form the perfluorinated allyl ether compound.

In another aspect, a method of making a perfluorinated allyl amine is described. The method comprising:
contacting a perfluorinated imine of Formula (III) with a metal or ammonium fluoride salt in an aprotic solvent to form an aza anion salt, where Formula (III) is R_{f}¹—N═CFR_{f}⁴ where (i) R_{f}¹ is a perfluorinated group selected from a perfluorinated alkyl group comprising 1-7 carbon atoms, a perfluorinated aryl group comprising a 5- or 6-membered ring, or combinations thereof, and optionally comprises one or more catenated heteroatoms selected from N or O and R_{f}⁴ is selected from F or a perfluorinated group selected from a perfluorinated alkyl group comprising 1-6 carbon atoms, and a perfluorinated aryl group comprising a 5- or 6-membered ring, or combinations thereof, and optionally comprises one or more catenated heteroatoms selected from N or O; or (ii) R_{f}¹ and R_{f}⁴ are bonded together to form a perfluorinated ring structure having 4-8 ring carbon atoms, optionally comprising at least one catenated O or N atom; and
contacting the aza anion salt with a perfluoroallylating reagent to form a perfluorinated allyl amine of formula (II)

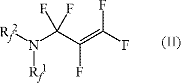

Where R_{f}¹ and R_{f}² are (i) independently selected from a perfluorinated alkyl group comprising 1-7 carbon atoms, a perfluorinated aryl group comprising a 5- or 6-membered ring, or combinations thereof, and optionally comprising one or more catenated heteroatoms selected from N or O; or (ii) bonded together to form a perfluorinated ring structure having 4-8 ring carbon atoms, optionally comprising at least one catenated O or N atom.

In another aspect, a polymerizable composition is described. The polymerizable composition comprising a fluorinated monomer and a perfluorinated allyl ether compound and perfluorinated allyl ether compound of formula (I)

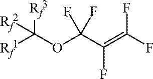

Where R_{f}¹ and R_{f}² are (i) independently selected from a perfluorinated alkyl group comprising 1-7 carbon atoms, a perfluorinated aryl group comprising a 5- or 6-membered ring, or combinations thereof and optionally comprising one or more catenated heteroatoms selected from N or O; or (ii) bonded together to form a perfluorinated ring structure having 4-8 ring carbon atoms, optionally comprising at least one catenated O or N atom; and
R_{f}³ is a perfluorinated alkyl group comprising 1-3 carbon atoms.

The above summary is not intended to describe each embodiment. The details of one or more embodiments of the invention are also set forth in the description below. Other features, objects, and advantages will be apparent from the description and from the claims.

### DETAILED DESCRIPTION

As used herein, the term
“a”, “an”, and “the” are used interchangeably and mean one or more; and
“and/or” is used to indicate one or both stated cases may occur, for example A and/or B includes, (A and B) and (A or B);
“alkyl” refers to a monovalent group that is a radical of an alkane, which is a saturated hydrocarbon. The alkyl group can be linear, branched, cyclic or combinations thereof;
“alkoxy” refers to a monovalent group of an alkyl group singularly bonded to an oxygen atom;
“aryl” refers to a monovalent group that is aromatic. The aryl has at least one aromatic ring. Any additional rings can be unsaturated, partially saturated, saturated, or aromatic;
“aralkyl” refers to a monovalent group that is an alkyl group substituted with an aryl group (e.g., as in a benzyl group);
“alkaryl” refers to a monovalent group that is an aryl substituted with an alkyl group (e.g., as in a tolyl group);
“catenated” means an atom other than carbon (for example, oxygen or nitrogen) that is bonded to at least two carbon atoms in a carbon chain (linear or branched or within a ring) so as to form an in-line carbon-heteroatom-carbon linkage;
“cure site” refers to functional groups, which may participate in crosslinking;
“interpolymerized” refers to monomers that are polymerized together to form the backbone of the polymer;
“monomer” is a molecule which can undergo polymerization which then form part of the essential structure of a polymer; and
“perfluorinated” means a group or a compound wherein all of the C-H bonds have been replaced with C-halogen bonds, with at least 50, 75, 90, 99 or even 100% of the C-halogens bonds being C-F bonds, preferably all of the C-halogens bonds are C-F bonds.

Also herein, recitation of ranges by endpoints includes all numbers subsumed within that range (e.g., 1 to 10 includes 1.4, 1.9, 2.33, 5.75, 9.98, etc.).

Also herein, recitation of “at least one” includes all numbers of one and greater (e.g., at least 2, at least 4, at least 6, at least 8, at least 10, at least 25, at least 50, at least 100, etc.).

As used herein, “comprises at least one of” A, B, and C refers to element A by itself, element B by itself, element C by itself, A and B, A and C, B and C, and a combination of all three.

The perfluorinated allyl ethers of the present disclosure are of the general formula (I)

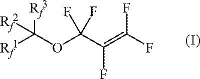

where:

R_{f}¹ and R_{f}² are (i) independently selected from a perfluorinated alkyl group comprising 1-7 carbon atoms, a perfluorinated aryl group comprising a 5- or 6-membered ring, or a combination thereof (such as an alkaryl or an aralkyl group) and optionally comprising one or more catenated heteroatoms selected from N or O; or (ii) bonded together to form a perfluorinated ring structure having 4-8 ring carbon atoms, optionally comprising at least one catenated O or N atom; and R_{f}³ is a perfluorinated alkyl group comprising 1-3 carbon atoms.

In one embodiment, R_{f}¹ and R_{f}² are independently selected from a linear or branched perfluoroalkyl group having at least 1, 2, or even 3 carbon atoms; and at most 4, 6, or even 8 carbon atoms, optionally comprising at least one catenated oxygen atom (or ether linkage) or nitrogen atom (or amine linkage).

In one embodiment, R_{f}¹ and/or R_{f}² are a combination of a perfluorinated alkyl group and a perfluorinated aryl group, optionally comprising at least one catenated O or N atom. For example, in one embodiment, R_{f}¹ and/or R_{f}² are a perfluorinated alkaryl or a perfluorinated aralkyl group, comprising from 4 to 8 carbon atoms and optionally comprising at least one catenated O or N atom.

Exemplary R_{f}¹ and/or R_{f}² groups include: —CF₃; —(CF₂)ₙCF₃ where n is 1, 2, 3, 4, 5, or 6; C(CF₃)₂CF₃; —CF(CF₃)CF₃; and —(CF₂)_{q}O(CF₂)ᵣCF₃, where q is an integer from 1-7, r is an integer from 0-6, where q+r is no more than 7, 6, 5, 4, 3, or even 2; a perfluorinated pyrrolyl, perfluorinated piperidinyl, a perfluorinated morpholinyl group, and combinations thereof.

In another embodiment, R_{f}¹ and R_{f}² are connected to form a ring structure moiety comprising a total of 4 to 8 carbon atoms in addition to optional catenary heteroatoms such as oxygen or nitrogen. The ring structure moiety may comprise a 4-, 5-, or 6- membered ring. In one embodiment, the ring structure is substituted with an alkyl or alkoxy group. Exemplary ringed structures include: 5-membered rings such as pyrroles, and 6-membered rings such pyridines.

Exemplary R_{f}³ groups include: —CF₃; —(CF₂)CF₃; —(CF₂)₂CF₃; and —CF(CF₃)CF₃.

Exemplary perfluorinated allyl ether compounds of the present disclosure include:

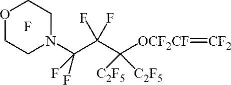

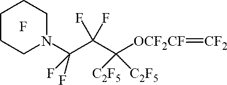

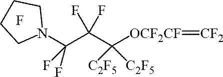

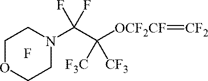

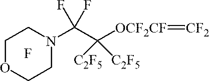

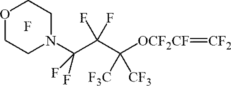

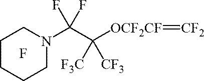

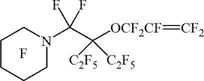

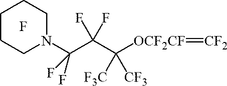

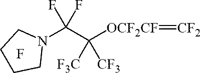

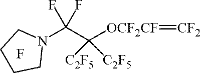

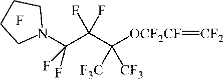

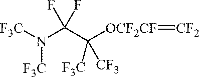

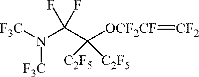

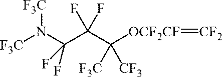

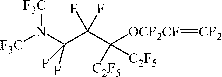

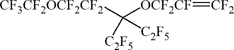

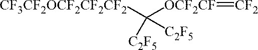

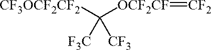

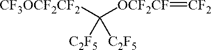

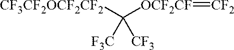

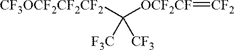

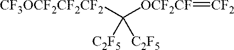

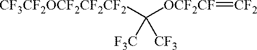

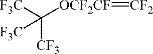

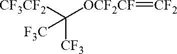

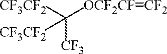

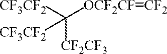

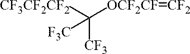

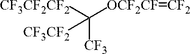

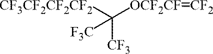

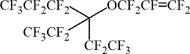

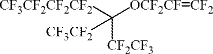

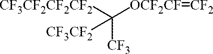

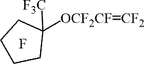

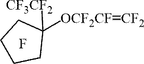

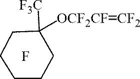

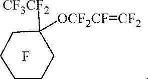

As used herein, the “F” within a ring structure means that each carbon within the ring structure is fluorinated.

In one embodiment, the perfluorinated allyl ether compound of the present disclosure can be prepared by contacting a perfluoroketone or perfluorinated acid fluoride with a metal or ammonium perfluorocarbanion salt in an aprotic solvent to form a perfluorinated tert-alkoxide salt. The perfluorinated tert-alkoxide salt can then be contacted with a perfluoroallylating reagent to form the perfluorinated allyl ether compound of formula (I).

In one embodiment, the metal or ammonium perfluorocarbanion salt may be generated by contacting tetrafluoroethylene, hexafluoropropylene, (CH₃)₃SiR_{f}³ or (CH₃CH₂)₃SiR_{f}³, wherein R_{f}³ is as defined above in Formula (I) with a fluoride salt to generate the perfluorinated tert-alkoxide salt intermediate. Fluoride salts are known in the art and can include metal fluoride and ammonium fluoride salts such as CsF, NaF, KF, RbF, MgF₂, CaF₂, tetraalkyl ammonium fluoride and combinations thereof. In one embodiment, the alkyl groups of the tetraalkyl ammonium group comprise 1, 2, 3, 4, 5, or even 6 carbon atoms. The alkyl groups of the tetraalkyl ammonium group may or may not be identical. Exemplary tetralkyl ammonium fluoride compounds include [N(CH₃)₄]F, [N(C₂H₅)₄]F, [N(C₃H₇)₄]F, and [N(C₄H₉)₄]F

In one embodiment, the perfluoroketones can be obtained from specialty chemical companies and suppliers such as Chemieliva Pharmaceutical Co., Ltd, Chongqing, China; or abcr GmbH, Karlsruhe, Germany. In one embodiment, the perfluorinated acid fluorides can be obtained from specialty chemical companies such as Exfluor, Research Corp., Round Rock, TX.

Exemplary perfluoroketone and perfluorinated acid fluoride include:

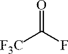

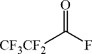

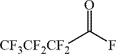

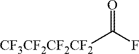

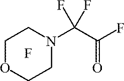

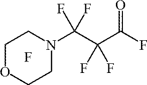

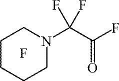

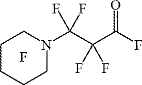

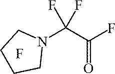

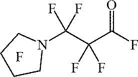

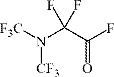 [SMILES: O=C(F)C(F)(F)N(C(F)(F)F)C(F)(F)F]

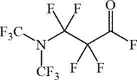 [SMILES: O=C(F)C(F)(F)C(F)(F)N(C(F)(F)F)C(F)(F)F]

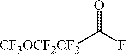

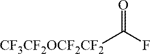

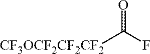

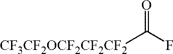

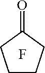

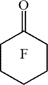

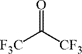

To make the perfluorinated allyl ethers of the present disclosure, the perfluoroketone or perfluorinated acid fluoride can be contacted with a metal or ammonium perfluorocarbanion salt in the presence of an aprotic solvent to form the perfluorinated tert-alkoxide salt. Polar aprotic solvents include, diglyme, tetraglyme, proglyme, tetrahydrofuran, cyclopentyl methyl ether, methyl-tert-butyl ether, dimethyl formamide, dimethylacetamide, N-methyl-2-pyrrolidone, sulfolane, nitriles (such as acetonitrile, adiponitrile, and benzonitrile), and dimethylsulfoxide. These polar aprotic solvents can be used individually or as a mixture.

In one embodiment, the metal or ammonium perfluorocarbanion salt is formed in situ in the presence of the perfluoroketone or perfluorinated acid fluoride. Such a reaction may be exothermic depending on the reagents, and as shown in the examples, care may be taken to keep the reaction temperature under 50, 40, 30, 25, or even 20° C.

The amount of metal or ammonium perfluorocarbanion salt used depends on whether a perfluoroketone or perfluorinated acid fluoride is used. In the case of the perfluoroketone, one equivalent of the carbanion reacts with one equivalent of the ketone. In one embodiment, the mole ratio of the perfluoroketone to the metal or ammonium perfluorocarbanion salt is typically less than 1 to 1, 0.85 to 1, or even less than 0.70 to 1. In the case of the perfluorinated acid fluoride, two equivalents of the carbanion reacts with one equivalent of the acid fluoride. In one embodiment, the mole ratio of the perfluorinated acid fluoride to the metal or ammonium perfluorocarbanion salt is typically less than 1 to 2, 0.85 to 2, or even less than 0.70 to 2.

The resulting perfluorinated tert-alkoxide salt is then contacted with a perfluoroallylating reagent to form the perfluorinated allyl ether compound of formula (I).

Exemplary perfluoroallylating reagent include perfluoroallyl fluorosulfate, perfluoroallyl iodide, perfluoroallyl chlorosulfate, perfluoroallyl triflate, and combinations thereof.

The mole ratio of the perfluorinated tert-alkoxide salt and the perfluoroallylating reagent is typically less than 1 to 1, 0.85 to 1, or even less than 0.70 to 1. Typically, lower temperatures are utilized during the allylation reaction, such as temperatures less than 50, 30, 25, 20, or even 10° C. In one embodiment, the temperature is at least 0, -5, -10, or even -20° C. Often the choice of temperature is selected based on the perfluorinated tert-alkoxide salt used and the ability to get the allylation reaction to go to completion.

In one embodiment, the method disclosed herein has a reaction yield for the perfluorinated allyl ether of at least 25, 30, 40, 50, 60, 70, or even 75%, based on the number of moles of perfluoroketone or perfluorinated acid used.

Using similar synthetic techniques, a perfluorinated allyl amine may be made by contacting a perfluorinated imine of Formula (III) with a metal or tetraalkyl ammonium fluoride salt in an aprotic solvent to form an aza anion salt and contacting the aza anion salt with a perfluoroallylating reagent to form a perfluorinated allyl amine.

The metal or tetraalkyl ammonium fluoride salt and the aprotic solvent used in the perfluorinated allyl amine synthesis can be the same as those disclosed above for the perfluorinated allyl ether synthesis.

The perfluorinated imine of Formula (III) is R_{f}¹—N═CFR_{f}⁴ where (i) R_{f}¹ is a perfluorinated group selected from a perfluorinated alkyl group comprising 1-7 carbon atoms, a perfluorinated aryl group comprising a 5- or 6-membered ring, or combinations thereof, and optionally comprises one or more catenated heteroatoms selected from N or O and R_{f}⁴ is selected from F or a perfluorinated group selected from a perfluorinated alkyl group comprising 1-6 carbon atoms, and a perfluorinated aryl group comprising a 5- or 6-membered ring, or combinations thereof, and optionally comprises one or more catenated heteroatoms selected from N or O; or (ii) R_{f}¹ and R_{f}⁴ are bonded together to form a perfluorinated ring structure having 4-8 ring carbon atoms, optionally comprising at least one catenated O or N atom.

R_{f}¹ can be defined the same as R_{f}¹ in formula (I).

In one embodiment, R_{f}⁴ is selected from F, or a linear, branched, or cyclic perfluoroalkyl group having at least 1, 2, or even 3 carbon atoms; and at most 4, 5, or even 6 carbon atoms, optionally comprising at least one catenated oxygen atom (or ether linkage) or nitrogen atom (or amine linkage).

In one embodiment, R_{f}⁴ is a combination of a perfluorinated alkyl group and a perfluorinated aryl group, optionally comprising at least one catenated O or N atom. For example, in one embodiment, R_{f}⁴ is a perfluorinated alkaryl or a perfluorinated aralkyl group, comprising from 4 to 7 carbon atoms and optionally comprising at least one catenated O or N atom.

Exemplary R_{f}⁴ groups include: —CF₃; —(CF₂)ₙCF₃ where n is 1, 2, 3, 4, or 5; C(CF₃)₂CF₃; —CF(CF₃)CF₃; and —(CF₂)_{q}O(CF₂)ᵣCF₃, where q is an integer from 1-6, r is an integer from 0-5, where q+r is no more than 6, 5, 4, 3, or even 2; a perfluorinated pyrrolyl, perfluorinated piperidinyl, a perfluorinated morpholinyl group, and combinations thereof.

In another embodiment, R_{f}¹ and R_{f}⁴ are connected to form a ring structure moiety comprising a total of 4 to 8 carbon atoms in addition to optional catenary heteroatoms such as oxygen or nitrogen. The ring structure moiety may comprise a 4-, 5-, or 6- membered ring. Exemplary ringed structures include: 5-membered rings such as pyrroles, and 6-membered rings such pyridines.

Exemplary perfluorinated imines of Formula (III) include:

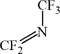

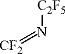

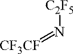

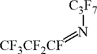

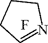

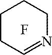

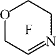

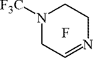

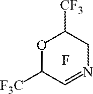

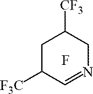

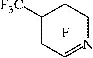

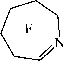

In one embodiment, the reaction of the perfluorinated imine of Formula (III) with the metal or tetraalkyl ammonium fluoride salt is an exothermic reaction and thus, care may be taken to keep the reaction temperature under 30, 25, 20, 10, 5 or even 0° C.

The mole ratio of the perfluorinated imine of Formula (III) to the metal or ammonium perfluorocarbanion salt is typically less than 1 to 1, 0.85 to 1, or even less than 0.70 to 1.

After forming the aza anion salt, the aza anion salt is then contacted with a perfluoroallylating reagent to form a perfluorinated allyl amine of formula (II) as shown below, where R_{f}¹ and R_{f}² are the same as those disclosed above in formula (I).

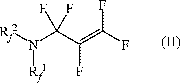

Exemplary perfluoroallylating reagent include perfluoroallyl fluorosulfate, perfluoroallyl iodide, perfluoroallyl chlorosulfate, perfluoroallyl triflate, and combinations thereof.

The mole ratio of the aza anion salt and the perfluoroallylating reagent is typically less than 1 to 1, 0.85 to 1, or even less than 0.70 to 1. Typically, lower temperatures are utilized during the allylation reaction, such as temperatures less than 50, 30, 25, 20, or even 10° C. In one embodiment, the temperature is at least 0, -5, -10, or even -20° C. Often the choice of temperature is selected based on the aza anion salt used and the desire to get the allylating reaction to go to completion.

In one embodiment, the method disclosed herein has a reaction yield for the perfluorinated allyl amine of at least 25, 30, 40, 50, 60, 70, or even 75%, based on the moles of the perfluorinated imine used.

Exemplary perfluorinated allyl amines that can be synthesized include:

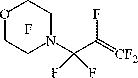

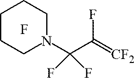

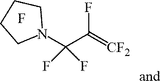

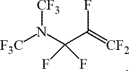

In one embodiment, the resulting perfluorinated compounds from the reactions above can be purified to isolate the desired perfluorinated allyl compound (such as the perfluorinated allyl ether or the perfluorinated allyl amine). Purification can be done by conventional means including distillation, absorption, extraction, chromatography and recrystallization. The purification can be done to isolate the perfluorinated allyl compound of the present disclosure from impurities, such as starting materials, byproducts, etc. The term “purified form” as used herein means the perfluorinated allyl compound of the present disclosure is at least 75, 80, 85, 90, 95, 98, or even 99 wt% pure.

Compounds comprising at least one perfluorinated allyl ether group made according to the methods of the present disclosure are useful, for example, in the preparation of fluoropolymers. For example, the perfluorinated allyl ether compound of formula (I) can be interpolymerized with at least one partially fluorinated or perfluorinated ethylenically unsaturated monomer represented by formula R^{a}CF═CR^{a}₂, wherein each R^{a} is independently fluoro, chloro, bromo, hydrogen, a fluoroalkyl group (e.g. perfluoroalkyl having from 1 to 8, 1 to 4, or 1 to 3 carbon atoms and optionally interrupted by one or more ether linkages), alkyl having up to 10 carbon atoms, alkoxy having up to 8 carbon atoms, or aryl having up to 8 carbon atoms. Examples of useful fluorinated monomers represented by formula R^{a}CF═CR^{a}₂ include vinylidene fluoride (VDF), tetrafluoroethylene (TFE), hexafluoropropylene (HFP), chlorotrifluoroethylene, 2-chloropentafluoropropene, trifluoroethylene, vinyl fluoride (VF), dichlorodifluoroethylene, 1,1-dichlorofluoroethylene, 1-hydropentafluoropropylene, 2-hydropentafluoropropylene, tetrafluoropropylene, and mixtures thereof. The perfluorinated allyl ether compound of formula (I) can be useful for preparing amorphous fluoropolymers, semi-crystalline thermoplastics, and non-melt processable fluoroplastics.

In some embodiments, the perfluorinated allyl ether compound of formula (I) can be copolymerized with TFE to form a non-melt processable fluoroplastic. The fluorinated allyl ether may be any of those described above. In a non-melt processable fluoroplastic, the perfluorinated allyl ether compound of formula (I) is included in the monomers for polymerization in an amount of up to about one percent by weight. TFE copolymers including a comonomer in an amount of up to about one percent by weight are referred to in the art as modified PTFE. Modified PTFE has such a high melt viscosity and/or low melt flow index (MFI) that it cannot be processed by conventional melt processing techniques such as extrusion, injection molding, or blow molding. In some embodiments, the fluoropolymer contains TFE units and units from the the perfluorinated allyl ether compound of formula (I) and no other comonomer units. The amount of the perfluorinated allyl ether comonomer units may be up to 1% by weight or up to 0.1% by weight. For example, the amount of the perfluorinated allyl ether comonomer units can be from 0.1 to 1 percent by weight or from 0.3 to 1 percent by weight, based on the total weight of the fluoropolymer (in which the comonomer units add up to give 100% by weight).

The molecular weights of certain fluoroplastics are often characterized by the melt viscosity or the melt flow index (MFI; e.g., 372° C./5 kg). In some embodiments, the non-melt-processable fluoropolymer made from the the perfluorinated allyl ether compound of formula (I) has a melt flow index (MFI) of 1.0 g / 10 min or less at 372° C. using a 5 kg load (MFI 372/5 of less than 1.0 g /10 min), in some embodiments, a melt flow index (372/5) of 0.1 g /10 minutes or less. In some embodiments, the non-melt-processable fluoropolymer has a melting point of at least 300° C., in some embodiments, at least 315° C., and typically within the range of 327 +/-10° C. In some embodiments, the non-melt-processable fluoropolymer has a melting point of at least 317° C., at least 319° C., or at least 321° C. The melting point of not melt-processable fluoropolymers differs when the material is molten for the first time and after subsequent melting. After the material has been molten once, the melting point in subsequent melting remains constant. The melting point as referred to herein is the melting point of previously molten material (i.e., the material was brought to the melting point, cooled below its melting point, and then melted again).

Modified PTFEs made with the perfluorinated allyl ether compound of formula (I) made by the methods disclosed herein can be useful, for example, for gaskets and inner liners for pipes and containers.

In some embodiments, the perfluorinated allyl ether compound of formula (I) can be copolymerized with TFE to form a fluorothermoplastic. In one embodiment, interpolymerized monomeric units of the perfluorinated allyl ether compound of formula (I) are present in the copolymer in an amount in a range from 0.01 mol% to 15 mol%, in some embodiments, 0.05 mol% to 10 mol%, and in some embodiments, 0.5 mol% to 5 mol%. In some embodiments, the copolymer of TFE and the perfluorinated allyl ether compound of formula (I) consists essentially of units derived from TFE and the perfluorinated allyl ether compound of formula (I). “Consisting essentially of” as used herein refers to the absence of other comonomers or the presence of units derived from other comonomers in an amount of less than one percent by weight, in some embodiments, less than 0.1 percent by weight. In some embodiments, the copolymer of TFE and the perfluorinated allyl ether compound of formula (I) further comprises at least one percent by weight, in some embodiments, up to 30, 20, 10, 6, 5, or 4 percent by weight of other units derived from compounds represented by formula R^{a}CF═CR^{a}₂ described above, non-fluorinated olefins (e.g., ethene or propene). In some embodiments, at least one of HFP, VDF, vinyl fluoride, chlorotrifluoroethylene, ethene, or propene is included in the monomers to make the fluorothermoplastic copolymer. In some embodiments, the fluorothermoplastic made from the compound comprising the perfluorinated allyl ether compound of formula (I) has a melt flow index (MFI) in a range from 0.5 g / 10 min to 100 g / 10 min at 372° C. using a 5 kg load (MFI 372/5 of in a range from 0.5 g /10 min to 100 g / 10 min). In some embodiments, the copolymer has a melting point of from 270° C. to 326° C. and a melt flow index (MFI at 372° C. and 5 kg load) of 0.5 to 19 grams / 10 minutes. In some embodiments, the copolymer has a melting point of from 200° C. to 290° C. and have a melt flow index (MFI at 372° C. and 5 kg load) of from 31 grams / 10 minutes to 100 grams / 10 minutes.

In some embodiments, the perfluorinated allyl ether compound of formula (I) can be copolymerized with TFE and HFP. The perfluorinated allyl ether compound of formula (I) may be any of those described above. Copolymers of TFE and HFP with or without other perfluorinated comonomers are known in the art as FEP’s (fluorinated ethylene propylene). In some embodiments, FEP-type fluorothermoplastics may be derived from copolymerizing 30 to 70 wt. % TFE, 10 to 30 wt. %, HFP, and 0.2 to 20 wt. % of the perfluorinated allyl ether compound of formula (I). These weight percentages are based on the weight of the polymer, and the comonomers add up to give 100% by weight. In some embodiments, units derived from the the perfluorinated allyl ether compound of formula (I) are present in the copolymer in a range from 0.2 percent by weight to 12 percent by weight, based on the total weight of the copolymer. In some embodiments, units derived from the perfluorinated allyl ether compound of formula (I) are present in a range from 0.5 percent by weight to 6 percent by weight, based on the total weight of the copolymer, with the total weight of the copolymer being 100% by weight. In some embodiments, units derived from the perfluorinated allyl ether compound of formula (I) are present in the copolymer according to the present disclosure in a range from 0.02 mole percent to 2 mole percent, based on the total amount of the copolymer. In some embodiments, units derived from the perfluorinated allyl ether compound of formula (I) are present in the copolymer in an amount up to 1.5 mole percent or up to 1.0 mole percent. In some embodiments, units derived from the perfluorinated allyl ether compound of formula (I) are present in the copolymer in an amount of at least 0.03 mole percent or 0.05 mole percent. The units derived from the perfluorinated allyl ether compound of formula (I) are present in the copolymer in a range from 0.02 mole percent to 2 mole percent, 0.03 mole percent to 1.5 mole percent, or 0.05 mole percent to 1.0 mole percent. Copolymers made according to the methods of the present disclosure may be made from any combination of units of the perfluorinated allyl ether compounds of formula (I) according to any of the above embodiments. The HFP may be present in a range from 5 wt. % to 22 wt. %, in a range from 10 wt. % to 17 wt. %, in a range from 11 wt. % to 16 wt. %, or in a range from 11.5 wt. % to 15.8 wt. %, based on the total weight of the copolymer, wherein the weight of the copolymer is 100% by weight. The copolymers made according to the methods of the present disclosure typically have a melting point between 220° C. to 285° C., in some embodiments, 235° C. to 275° C., 240° C. to 275° C., or 245° C. to 265° C. In some embodiments, the copolymer prepared from the compound(s) comprising the perfluorinated allyl ether compound of formula (I), TFE, and HFP has an MFI at 372° C. and 5 kg load of 30 ± 10 grams per 10 minutes. In some embodiments, the copolymer prepared from the perfluorinated allyl ether compound of formula (I), TFE, and HFP has an MFI at 372° C. and 5 kg load of 30 ± 5 grams per 10 minutes or 30 ± 3 grams per 10 minutes. In some embodiments, the copolymer prepared from the perfluorinated allyl ether compound of formula (I), TFE, and HFP has an MFI at 372° C. and 5 kg load in a range from 1 gram per 10 minutes to 19 grams per 10 minutes. In some embodiments, this copolymer has an MFI in a range from 1 gram per 10 minutes to 15 grams per 10 minutes or in a range from 1 gram per 10 minutes to 10 grams per 10 minutes.

FEPs made with one or more compounds comprising the perfluorinated allyl ether compound of formula (I) made by the methods disclosed herein can be useful, for example, for electrical insulation in Local Area Networks (LAN).

In some embodiments, the perfluorinated allyl ether compound of formula (I) disclosed herein can be used to make amorphous fluoropolymers. Amorphous fluoropolymers typically do not exhibit a melting point and exhibit little or no crystallinity at room temperature. Useful amorphous fluoropolymers can have glass transition temperatures below room temperature or up to 280° C. Suitable amorphous fluoropolymers can have glass transition temperatures in a range from -60 ℃ up to 280° C., -60° C. up to 250° C., from -60° C. to 150° C., from -40° C. to 150° C., from -40° C. to 100° C., or from -40° C. to 20° C.

In some embodiments, polymerized units derived from the perfluorinated allyl ether compound of formula (I) are present in the amorphous fluoropolymer at up to 50 mole percent of the fluoropolymer, in some embodiments up to 30 mole percent or up to 10 mole percent.

In some embodiments, the amorphous fluoropolymer contains cure sites, which facilitate cross-linking of the fluoropolymer in appropriate cure systems. These cure sites comprise at least one of iodine, bromine, and/or nitrile. The fluoropolymer may be polymerized in the presence of a chain transfer agent and/or cure site monomer to introduce cure sites into the polymer. Such cure site monomers and chain transfer agents are known in the art. Exemplary chain transfer agents include: an iodo-chain transfer agent, a bromo-chain transfer agent, or a chloro-chain transfer agent. For example, suitable iodo-chain transfer agent in the polymerization include the formula of RIₓ, where (i) R is a perfluoroalkyl or chloroperfluoroalkyl group having 3 to 12 carbon atoms; and (ii) x = 1 or 2. The iodo-chain transfer agent may be a perfluorinated iodo-compound. Exemplary iodo-perfluoro-compounds include 1,3-diiodoperfluoropropane, 1,4-diiodoperfluorobutane, 1, 6-diiodoperfluorohexane, 1,8-diiodoperfluorooctane, 1,10-diiodoperfluorodecane, 1,12-diiodoperfluorododecane, 2-iodo-1,2-dichloro-1,1,2-trifluoroethane, 4-iodo-1,2,4-trichloroperfluorobutan, and mixtures thereof. In some embodiments, the iodo-chain transfer agent is of the formula I(CF₂)ₙ—O—R_{f}—(CF₂)ₘI, wherein n is 1, 2, 3, 4, 5, 6, 7, 8, 9, or 10, m is 1, 2, 3, 4, 5, 6, 7, 8, 9, or 10 and R_{f} is a partially fluorinated or perfluorinated alkylene segment, which can be linear or branched and optionally comprises at least one catenated ether linkage. Exemplary compounds include: I—CF₂—CF₂—O—CF₂—CF₂—I, I—CF(CF₃)—CF₂—O—CF₂—CF₂—I, I—CF₂—CF₂—O—CF(CF₃)—CF₂—O—CF₂—CF₂—I, I—(CF(CF₃)—CF₂—O)₂—CF₂—CF₂—I,, I—CF₂—CF₂—O—(CF₂)₂—O—CF₂—CF₂—I, I—CF₂—CF₂—O—(CF₂)₃—O—CF₂—CF₂—I, and I—CF₂—CF₂—O—(CF₂)₄—O—CF₂—CF₂—I, I—CF₂—CF₂—CF₂—O—CF₂—CF₂—I, and I—CF₂—CF₂—CF₂—O—CF(CF₃)—CF₂—O—CF₂—CF₂—I. In some embodiments, the bromine is derived from a brominated chain transfer agent of the formula: RBrₓ, where (i) R is a perfluoroalkyl or chloroperfluoroalkyl group having 3 to 12 carbon atoms; and (ii) x = 1 or 2. The chain transfer agent may be a perfluorinated bromo-compound.

In one embodiment, the cure site monomers may be of the formula: (a) CX₂=CX(Q′), wherein: (i) X each is independently H or F; and (ii) Q′ is I, Br, R_{f}⁵ —I or R_{f}⁵ —Br wherein R_{f}⁵ =a perfluorinated or partially fluorinated alkylene group optionally containing ether linkages or (b)

Y(CF₂)_{q}Y, wherein: (i) Y is independently selected from Br or I or Cl and (ii) q=1-6. In addition, non-fluorinated bromo-or iodo-olefins, e.g., vinyl iodide and allyl iodide, can be used. Exemplary cure site monomers include: CH₂═CHI, CF₂═CHI, CF₂═CFI, CH₂═CHCH₂I, CF₂═CFCF₂I, ICF₂CF₂CF₂CF₂I, CH₂═CHCF₂CF₂I, CF₂═CFCH₂CH₂I, CF₂═CFCF₂CF₂I, CH₂═CH(CF₂)₆CH₂CH₂I, CF₂═CFOCF₂CF₂I, CF₂═CFOCF₂CF₂CF₂I, CF₂═CFOCF₂CF₂CH₂I, CF₂═CFCF₂OCH₂CH₂I, CF₂═CFO(CF₂)₃—OCF₂CF₂I, CH₂═CHBr, CF₂═CHBr, CF₂═CFBr, CH₂═CHCH₂Br, CF₂═CFCF₂Br, CH₂═CHCF₂CF₂Br, CF₂═CFOCF₂CF₂Br, CF₂═CFCl, I—CF₂—CF₂CF₂—O—CF═CF₂, I—CF₂—CF₂CF₂—O—CF₂CF═CF₂, I—CF₂—CF₂—O—CF₂—CF═CF₂, I—CF(CF₃)—CF₂—O—CF═CF₂, I—CF(CF₃)—CF₂—O—CF₂—CF═CF₂, I—CF₂—CF₂—O—CF(CF₃)—CF₂—O—CF═CF₂, I—CF₂—CF₂—O—CF(CF₃)—CF₂—O—CF₂—CF═CF₂, I—CF₂—CF₂—(O—(CF(CF₃)—CF₂)₂—O—CF═CF₂, I—CF₂—CF₂—(O—(CF(CF₃)—CF₂)₂—O—CF₂—CF═CF₂, Br—CF₂—CF₂—O—CF₂—CF═CF₂, Br—CF(CF₃)—CF₂—O—CF═CF₂, I—CF₂—CF₂—CF₂—O—CF(CF₃)—CF₂—O—CF═CF₂, I—CF₂—CF₂—CF₂—O—CF(CF₃)—CF₂—O—CF₂—CF═CF₂, I—CF₂—CF₂—CF₂—(O—(CF(CF₃)—CF₂)₂—O—CF═CF₂, I—CF₂—CF₂—CF₂—O—(CF(CF₃)—CF₂—O)₂—CF₂—CF═CF₂, Br—CF₂—CF₂—CF₂—O—CF═CF₂, Br—CF₂—CF₂—CF₂—O—CF₂—CF═CF₂, I—CF₂—CF₂—O—(CF₂)₂—O—CF═CF₂, I—CF₂—CF₂—O—(CF₂)₃—O—CF═CF₂, I—CF₂—CF₂—O—(CF₂)₄—O—CF═CF₂, I—CF₂—CF₂—O—(CF₂)₂—O—CF₂—CF═CF₂, I—CF₂—CF₂—O—(CF₂)₃—O—CF₂—CF═CF₂, I—CF₂—CF₂—O—(CF₂)₂—O—CF(CF₃)CF₂—O—CF₂═CF₂, I—CF₂—CF₂—O—(CF₂)₂—O—CF(CF₃)CF₂—O—CF₂—CF₂═CF₂, Br—CF₂—CF₂—O—(CF₂)₂—O—CF═CF₂, Br—CF₂—CF₂—O—(CF₂)₃—O—CF═CF₂, Br—CF₂—CF₂—O—(CF₂)₄—O—CF═CF₂, and Br—CF₂—CF₂—O—(CF₂)₂—O—CF₂—CF═CF₂.

Examples of nitrile containing cure site monomers correspond to the following formula— CF₂═CF—CF₂—O—Rf⁶—CN; CF₂═CFO(CF₂)ᵣCN; CF₂═CFO[CF₂CF(CF₃)O]ₚ(CF₂)ᵥOCF(CF₃)CN; and CF₂═CF[OCF₂CF(CF₃)]ₖO(CF₂)ᵤCN; wherein, r represents an integer of 2 to 12; p represents an integer of 0 to 4; k represents 1 or 2; v represents an integer of 0 to 6; u represents an integer of 1 to 6; and R_{f}⁶ is a perfluoroalkylene or a bivalent perfluoroether group. Specific examples of nitrile containing fluorinated monomers include, but are not limited to, perfluoro (8-cyano-5-methyl-3,6-dioxa-1-octene), CF₂═CFO(CF₂)₅CN, and CF₂═CFO(CF₂)₃OCF(CF₃)CN.

In one embodiment, the fluoropolymer of the present disclosure comprises at least 0.1, 0.5, 1, 2, or even 2.5 wt% of iodine, bromine, and/or nitrile groups versus the total weight of fluoropolymer. In one embodiment, the fluoropolymer comprises no more than 3, 5, or even 10 wt% of iodine, bromine, and/or nitrile groups versus the total weight of the fluoropolymer.

In one embodiment, the cure site monomer can be perfluorinated to ensure adequate thermal stability of the resulting elastomer. Fluoropolymers containing a Br cure site, an I cure site, a nitrile cure site, a carbon-carbon double bond, and combinations thereof may be cured using peroxides, for example. However, in some cases in which multiple, different cure sites are present a dual cure system or a multi cure system may be useful. Other suitable cure systems that may be useful include bisphenol curing systems or triazine curing systems. Useful amounts of the cure site monomers include 0.01 mol % to 1 mol %, based on total moles of monomer incorporated into the polymer may be used. In some embodiments, at least 0.02, 0.05, or even 0.1 mol % of a cure site monomer is used and at most 0.5, 0.75, or even 0.9 mol % of a cure site monomer is used based on the total moles of monomer incorporated into the amorphous fluoropolymer.

If the amorphous fluoropolymer is perhalogenated, in some embodiments perfluorinated, typically at least 50 mole percent (mol %) of its interpolymerized units are derived from TFE and/or CTFE, optionally including HFP. The balance of the interpolymerized units of the amorphous fluoropolymer (e.g., 10 to 50 mol %) is made up of the perfluorinated allyl ether compound of formula (I), and, in some embodiments, a cure site monomer. If the fluoropolymer is not perfluorinated, it typically contains from about 5 mol % to about 90 mol % of its interpolymerized units derived from TFE, CTFE, and/or HFP; from about 5 mol % to about 90 mol % of its interpolymerized units derived from VDF, ethylene, and/or propylene; up to about 40 mol % of its interpolymerized units derived from the perfluorinated allyl ether compound of formula (I); and from about 0.1 mol % to about 5 mol %, in some embodiments from about 0.3 mol % to about 2 mol %, of a cure site monomer.

In embodiments in which the methods disclosed herein comprise combining the compound comprising the perfluorinated allyl ether compound of formula (I) with at least one partially fluorinated or perfluorinated ethylenically unsaturated monomer represented by formula R^{a}CF=CR^{a}₂, the reaction can be carried out by free-radical polymerization. Conveniently, in some embodiments, the methods of making the copolymer disclosed herein includes radical aqueous emulsion polymerization.

In some embodiments of the methods of making the copolymer according to the present disclosure, a water-soluble initiator (e.g., potassium permanganate or a peroxy sulfuric acid salt) can be useful to start the polymerization process. Salts of peroxy sulfuric acid, such as ammonium persulfate or potassium persulfate, can be applied either alone or in the presence of a reducing agent, such as bisulfites or sulfinates (e.g., fluorinated sulfinates disclosed in U.S. Pat. Nos. 5,285,002 and 5,378,782, both to Grootaert) or the sodium salt of hydroxy methane sulfinic acid (sold under the trade designation “RONGALIT”, BASF Chemical Company, New Jersey, USA). The choice of initiator and reducing agent, if present, will affect the end groups of the copolymer. The concentration range for the initiators and reducing agent can vary from 0.01% to 5% by weight based on the aqueous polymerization medium.

Typical chain-transfer agents like H₂, lower alkanes, alcohols, ethers, esters, and methylene fluoride may be useful in the preparation of the copolymer in some embodiments of the method according to the present disclosure. Termination primarily via chain-transfer results in a polydispersity of about 2.5 or less. In some embodiments of the method according to the present disclosure, the polymerization is carried out without any chain-transfer agents. A lower polydispersity can sometimes be achieved in the absence of chain-transfer agents. Recombination typically leads to a polydispersity of about 1.5 for small conversions.

Useful polymerization temperatures can range from 40° C. to 150° C. Typically, polymerization is carried out in a temperature range from 40 ℃ to 120° C., 70° C. to 100° C., or 80° C. to 90° C. The polymerization pressure is usually in the range of 0.8 MPa to 2.5 MPa, 1 MPa to 2.5 MPa, and in some embodiments is in the range from 1.0 MPa to 2.0 MPa. Fluorinated monomers such as HFP can be precharged and fed into the reactor as described, for example, in Modern Fluoropolymers, ed. John Scheirs, Wiley & Sons, 1997, p. 241.

In some embodiments, perfluorinated or partially fluorinated emulsifiers may be useful. Generally these fluorinated emulsifiers are present in a range from about 0.02% to about 3% by weight with respect to the polymer. Polymer particles produced with a fluorinated emulsifier typically have an average diameter, as determined by dynamic light scattering techniques, in range of about 10 nanometers (nm) to about 300 nm, and in some embodiments in range of about 50 nm to about 200 nm. Examples of suitable emulsifiers include perfluorinated and partially fluorinated emulsifier having the formula [R_{f}⁸—O—L—COO⁻]ᵢXⁱ⁺ wherein L represents a linear partially or fully fluorinated alkylene group or an aliphatic hydrocarbon group, R_{f}⁸represents a linear partially or fully fluorinated aliphatic group or a linear partially or fully fluorinated aliphatic group interrupted with one or more oxygen atoms, Xⁱ⁺ represents a cation having the valence i and i is 1, 2 or 3. (See, e.g., U.S. Pat. No. 7,671,112 to Hintzer et al.). Additional examples of suitable emulsifiers also include perfluorinated polyether emulsifiers having the formula CF₃—(OCF₂)ₓ—O—CF₂—X′, wherein x has a value of 1 to 6 and X′ represents a carboxylic acid group or salt thereof, and the formula CF₃—O—(CF₂)₃—(OCF(CF₃)—CF₂)_{y}—O—L—Y′ wherein y has a value of 0, 1, 2 or 3, L represents a divalent linking group selected from —CF(CF₃)—, —CF₂—, and—CF₂CF₂—, and Y′ represents a carboxylic acid group or salt thereof. (See, e.g., U.S. Pat. Publ. No. 2007/0015865 to Hintzer et al.). Other suitable emulsifiers include perfluorinated polyether emulsifiers having the formula R_{f}⁹—O(CF₂CF₂O)ₓCF₂COOA wherein R_{f}⁹ is C_{b}F_{(2b+1)}; where b is 1 to 4, A is a hydrogen atom, an alkali metal or NH₄, and x is an integer of from 1 to 3. (See, e.g., U.S. Pat. Publ. No. 2006/0199898 to Funaki et al.). Suitable emulsifiers also include perfluorinated emulsifiers having the formula F(CF₂)_{b}O(CF₂CF₂O)ₓCF₂COOA wherein A is a hydrogen atom, an alkali metal or NH₄, b is an integer of from 3 to 10, and x is 0 or an integer of from 1 to 3. (See, e.g., U.S. Pat. Publ. No. 2007/0117915 to Funaki et al.). Further suitable emulsifiers include fluorinated polyether emulsifiers as described in U.S. Pat. No. 6,429,258 to Morgan et al. and perfluorinated or partially fluorinated alkoxy acids and salts thereof wherein the perfluoroalkyl component of the perfluoroalkoxy has 4 to 12 carbon atoms, or 7 to 12 carbon atoms. (See, e.g., U.S. Pat. No. 4,621,116 to Morgan). Suitable emulsifiers also include partially fluorinated polyether emulsifiers having the formula [R_{f}¹⁰—(O)ₜ—CHF—(CF₂)ₓ—COO—]ᵢXⁱ⁺ wherein R_{f}¹⁰ represents a partially or fully fluorinated aliphatic group optionally interrupted with one or more oxygen atoms, t is 0 or 1 and x is 0 or 1, Xⁱ⁺ represents a cation having a valence i and i is 1, 2 or 3. (See, e.g., U.S. Pat. Publ. No. 2007/0142541 to Hintzer et al.). Further suitable emulsifiers include perfluorinated or partially fluorinated ether-containing emulsifiers as described in U.S. Pat. Publ. Nos. 2006/0223924, 2007/0060699, and 2007/0142513 each to Tsuda et al. and 2006/0281946 to Morita et al. Conveniently, in some embodiments, the method of making the copolymer according to the present disclosure may be conducted in the absence of any of these emulsifiers or any combination thereof, for example, using the methods found in U.S. Pat. Publ. No. 2007/0149733 (Otsuka).

If fluorinated emulsifiers are used, the emulsifiers can be removed or recycled from the fluoropolymer latex, if desired, as described in U.S. Pat. Nos. 5,442,097 to Obermeier et al., 6,613,941 to Felix et al., 6,794,550 to Hintzer et al., 6,706,193 to Burkard et al., and 7,018,541 to Hintzer et al.

The polymerization can be carried out without adding any perfluorinated alkanoic acids, in particular perfluorinated alkanoic acids with 6 to 14 carbon atoms, and in particular with 8 carbon atoms (perfluorinated octanoic acid (PFOA)) to the reaction mixture. Perfluorinated alkanoic acids represented by formula Rf—(CF₂)ₙ—A, wherein Rf is a perfluorinated alkyl radical that only contains F and C atoms, n is an integer of 5 to 14 and A is an acid anion salt, for example a —COO⁻X wherein X is H⁺, or a cationic salt such as NH₄⁺ or Na⁺ another metal salt, have become under increased scrutiny because of their environmental persistence and bioaccumulation. Therefore, their use is avoided. However, even if no such emulsifiers are used in the preparation of the polymers, they may be generated in situ in certain reactions. As another advantage, the copolymers made by the methods of the present disclosure may have a very low extractable amount of perfluorinated alkanoic acids, for example amounts of less than 100 ppb based on the weight of C₆-C₁₂, preferably C₆ to C₁₄ perfluorinated alkanoic acids, and may have an amount of extractable octanoic acid (C₈) of less than 50 ppb, preferably less than 30 ppb – based on the weight of the polymer.

In some embodiments, the obtained copolymer may be purified by at least one of anion- or cation-exchange processes to remove functional comonomers, anions, and/or cations before coagulation or spray drying (described below). As used herein, the term “purify” refers to at least partially removing impurities, regardless of whether the removal is complete. The obtained copolymer dispersion after aqueous emulsion polymerization and optional ion-exchange purification can be used as is or, if higher solids are desired, can be upconcentrated.

To coagulate the obtained copolymer latex, any coagulant which is commonly used for coagulation of a fluoropolymer latex may be used, and it may, for example, be a water-soluble salt (e.g., calcium chloride, magnesium chloride, aluminum chloride or aluminum nitrate), an acid (e.g., nitric acid, hydrochloric acid or sulfuric acid), or a water-soluble organic liquid (e.g., alcohol or acetone). The amount of the coagulant to be added may be in a range of 0.001 to 20 parts by mass, for example, in a range of 0.01 to 10 parts by mass per 100 parts by mass of the latex. Alternatively or additionally, the latex may be frozen for coagulation or mechanically coagulated, for example, with a homogenizer as described in U.S. Pat. No. 5,463,021 (Beyer et al.). Alternatively or additionally, the latex may be coagulated by adding polycations. It may also be useful to avoid acids and alkaline earth metal salts as coagulants to avoid metal contaminants. To avoid coagulation altogether and any contaminants from coagulants, spray drying the latex after polymerization and optional ion-exchange purification may be useful to provide solid copolymer.

A coagulated copolymer can be collected by filtration and washed with water. The washing water may, for example, be ion-exchanged water, pure water, or ultrapure water. The amount of the washing water may be from 1 to 5 times by mass to the copolymer, whereby the amount of the emulsifier attached to the copolymer can be sufficiently reduced by one washing.

In some embodiments of the methods of making the copolymer or ionomer according to the present disclosure, radical polymerization also can be carried out by suspension polymerization. Suspension polymerization will typically produce particle sizes up to several millimeters.

Post-fluorination with fluorine gas is commonly used to cope with unstable end groups and any concomitant degradation. Post-fluorination of the fluoropolymer can convert —COOH, amide, hydride, —COF, and other nonperfluorinated end groups or —CF═CF₂ to —CF₃ end groups. The post-fluorination may be carried out in any convenient manner. The post-fluorination can be conveniently carried out with nitrogen/fluorine gas mixtures in ratios of 75 - 90 : 25 - 10 at temperatures between 20° C. and 250° C., in some embodiments in a range of 150° C. to 250° C. or 70° C. to 120° C., and pressures from 100 KPa to 1000 KPa. Reaction times can range from about four hours to about 16 hours. Under these conditions, most unstable carbon-based end groups are removed, whereas any —SO₂X groups mostly survive and are converted to —SO₂F groups. In some embodiments, post-fluorination is not carried out when non-fluorinated monomers described above are used as monomers in the polymerization.

In one embodiment, the copolymer prepared according to the methods of the present disclosure is substantially free (i.e., comprises less than 0.5, 0.1, 0.05 wt % or even no) of —SO₂—units, based on the total weight of the fluoropolymer.

The copolymer prepared according to the methods of the present disclosure can be essentially free of copolymerized units derived from a perfluorinated alkyl vinyl ether [e.g., perfluoromethyl vinyl ether (CF₂═CFOCF₃), perfluoroethyl vinyl ether (CF₂═CFOCF₂CF₃), and perfluoropropyl vinyl ether (CF₂═CFOCF₂CF₂CF₃)] and perfluoroalkoxyalkyl vinyl ethers. “Essentially free” as used herein is referred to an amount of 0 to 0.9 % by weight, in some embodiments from 0 to 0.1 % by weight. The copolymers prepared by the methods of the present disclosure can be prepared without using any vinyl ethers, although minor amounts of vinyl ethers present as impurities may be tolerated. Examples of perfluoroalkoxyalkyl vinyl ethers that may be avoided include CF₂═CFOCF₂OCF₃, CF₂═CFOCF₂OCF₂CF₃, CF₂═CFOCF₂CF₂OCF₃, CF₂═CFOCF₂CF₂CF₂OCF₃, CF₂═CFOCF₂CF₂CF₂CF₂OCF₃, CF₂═CFOCF₂CF₂OCF₂CF₃, CF₂═CFOCF₂CF₂CF₂OCF₂CF₃, CF₂═CFOCF₂CF₂CF₂CF₂OCF₂CF₃, CF₂═CFOCF₂CF₂OCF₂OCF₃, CF₂═CFOCF₂CF₂OCF₂CF₂OCF₃, CF₂═CFOCF₂CF₂OCF₂CF₂CF₂OCF₃, CF₂═CFOCF₂CF₂OCF₂CF₂CF₂CF₂OCF₃, CF₂═CFOCF₂CF₂OCF₂CF₂CF₂CF₂CF₂OCF₃, CF₂═CFOCF₂CF₂(OCF₂)₃OCF₃, CF₂═CFOCF₂CF₂(OCF₂)₄OCF₃, CF₂═CFOCF₂CF₂OCF₂OCF₂OCF₃, CF₂═CFOCF₂CF₂OCF₂CF₂CF₃, CF₂═CFOCF₂CF₂OCF₂CF₂OCF₂CF₂CF₃, CF₂═CFOCF₂CF(CF₃)—O—C₃F₇ (PPVE—2), CF₂═CF(OCF₂CF(CF₃))₂—O—C₃F₇ (PPVE—3), and CF₂═ CF(OCF₂CF(CF₃))₃—O—C₃F₇ (PPVE—4).

Vinyl ethers can undergo termination reactions (e.g. cleavage of vinyl ether) during polymerization, in particular at higher temperatures, and creating unstable carboxy-endgroups.

The copolymer prepared according to the methods of the present disclosure can be essentially free of copolymerized units derived from a perfluorinated alkyl allyl ethers and perfluoroalkyloxyalkyl allyl ethers not according to Formula (I). “Essentially free” as used herein is referred to an amount of 0 to 0.9 % by weight, in some embodiments from 0 to 0.1 % by weight. Examples of perfluorinated alkyl allyl ethers and perfluoroalkyloxyalkyl allyl ethers that may be avoided include linear perfluorinated alkyl allyl ethers and perfluoroalkyloxyalkyl allyl ethers such as CF₂═CFCF₂O(CF₂)ᵤCF₃, where u is an integer from 0-7, CF₂═CFCF₂O(CF₂)_{y}O(CF₂)_{z}CF₃, y is 1-3 and z is 0-4 .

### EXAMPLES

Unless otherwise noted, all parts, percentages, ratios, etc. in the examples and the rest of the specification are by weight, and all reagents used in the examples were obtained, or are available, from general chemical suppliers such as, for example, Sigma-Aldrich Corp., Saint Louis, MO, or may be synthesized by conventional methods.

The following abbreviations are used in this section: mL=milliliter, min=minutes, h=hours, g=gram, mol=mole, mmol=millimole, °C = degrees Celsius.

TABLE 1

Materials List

| | |
|---|---|
| Material | Description |
| CsF | Cesium fluoride, commercially available from Alfa Aesar, Ward Hill, MA |
| KF | Potassium fluoride, commercially available from Sigma Aldrich Corp., St. Louis, MO |
| Diglyme | Diethylene glycol dimethyl ether, commercially available from Sigma Aldrich Corp. |
| Tetraglyme | Tetraethylene glycol dimethyl ether, commercially available from Alfa Aesar |
| PFAFS | Perfluoroallyl fluorosulfate, Commercially available from Synquest Laboratories, Inc., Alachua, FL |
| Perfluoroallyl iodide | Commercially available from Synquest Laboratories, Inc. |
| 2,2,3,3,5,6,6-heptafluoro-1,4-oxazine | Can be prepared as described in V. A. Petrov, et al., J. Fluorine Chem 1996, 77, pages 139-142. |
| 1,1-difluoro-N-(trifluoromethyl)methanimine | Can be prepared as described in A. F. Gontar et al. Izvestiya Akademii Nauk SSSR, Seriya Khimicheskaya 1984, 1874. |
| 18-Crown-6 | Commercially available from Oakwood Chemical, Gainesville, FL |
| Perfluoropropionyl fluoride | 2,2,3,3,3-Pentafluoropropionyl fluoride, provided by 3M Co., Maplewood, MN |
| Hexafluoroacetone | Commercially available from Synquest Laboratories, Inc. |
| TMSCF₃ | Trifluoromethyltrimethylsilane, commercially available from Oakwood Chemical |
| TMSCF₂CF₃ | Pentafluoroethyltrimethylsilane, commercially available from Oakwood Chemical |
| DMF | N,N-Dimethylformamide, commercially available from Sigma Aldrich Corp. |
| Acetonitrile | Commercially available from Sigma Aldrich Corp. |

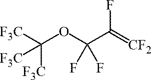

### Examples 1A and 1B: Preparation of 1,1,2,3,3-Pentafluoro-3-((1,1,1,3,3,3-Hexafluoro-2-(Trifluoromethyl)Propan-2-yl)Oxy)Prop-1-Ene

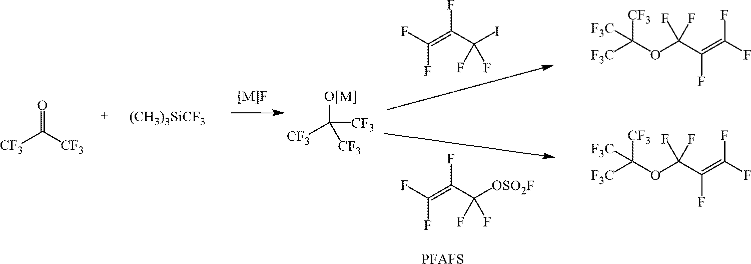

Step 1: To a 600 mL stainless steel reaction vessel were added potassium fluoride (18.8 g, 324 mmol) and acetonitrile (125 mL). The reactor was sealed and then evacuated and backfilled with nitrogen three times. After the final evacuation, stirring commenced and hexafluoroacetone (50.1 g, 302 mmol) was added at a rate which avoided a temperature increase above 30° C. The resultant mixture was stirred and once the reaction temperature fell to 25° C., TMSCF₃ (42.9 g, 302 mmol) was added from a stainless steel cylinder pressurized with argon gas at a rate which did not allow for the reaction mixture temperature to rise above 30° C. The reaction mixture slowly cooled to 25° C. and was allowed to stir at this temperature overnight. The resultant reaction mixture was then transferred to a round-bottom flask and concentrated under reduced pressure, affording potassium 1,1,1,3,3,3-hexafluoro-2-(trifluoromethyl)propan-2-olate as a white solid (60.5 g, 73% yield). This intermediate was used in the next step without additional purification.

Step 2A (Preparation of Example 1A): A 3-neck round-bottom flask equipped with a magnetic stir bar, temperature probe, and reflux condenser was charged with potassium 1,1,1,3,3,3-hexafluoro-2-(trifluoromethyl)propan-2-olate (30.1 g, 110 mmol). The flask was evacuated and backfilled with nitrogen three times followed by the addition of tetraglyme (70 mL). With stirring, the mixture was cooled to 0-5° C. followed by the dropwise addition of perfluoroallyl iodide (28.2 g, 104 mmol) at a rate which avoided reaction temperature increases above 8° C. The resultant reaction mixture was allowed to stir at the same temperature for 30 minutes. GC-FID (gas chromatography- flame ionization detection) analysis indicated complete conversion of the perfluoroallyl iodide. Water (100 mL) was slowly added to the reaction mixture. The resultant mixture was transferred to a separatory funnel and the fluorochemical layer was collected and purified via fractional distillation (90° C., 740 mm/Hg) affording the desired 1,1,2,3,3-pentafluoro-3-((1,1,1,3,3,3-hexafluoro-2-(trifluoromethyl)propan-2-yl)oxy)prop-1-ene (31.1 g, 77% isolated yield) as a colorless liquid. The identity of the isolated composition was confirmed by GC-MS (gas chromatography- mass spectrometry) analysis.

Step 2B (Preparation of Example 1B): To a 3-neck round-bottom flask equipped with a magnetic stir bar, reflux condenser, and temperature probe was charged potassium 1,1,1,3,3,3-hexafluoro-2-(trifluoromethyl)propan-2-olate (25.4 g, 92.7 mmol). The flask was evacuated and backfilled with nitrogen three times followed by the addition of tetraglyme (100 mL). The stirring mixture was then cooled to 0° C. followed by the dropwise addition of PFAFS (21.3 g, 92.7 mmol) at a rate which did not allow for the reaction temperature to rise above 5° C. After complete addition, the resultant reaction mixture was allowed to stir for 2 h at 0-5° C. Stirring was stopped and separation of the top, organic layer afforded a fluorochemical mixture (15.8 g) for which GC-FID analysis indicated complete conversion of PFAFS and formation of the desired 1,1,2,3,3-pentafluoro-3-((1,1,1,3,3,3-hexafluoro-2-(trifluoromethyl)propan-2-yl)oxy)prop-1-ene (35% yield based on GC). Fractional distillation (90° C., 740 mm/Hg) afforded the desired 1,1,2,3,3-pentafluoro-3-((1,1,1,3,3,3-hexafluoro-2-(trifluoromethyl)propan-2-yl)oxy)prop-1-ene (10.1 g, 29.8% isolated yield) as a colorless liquid. The identity of the isolated composition was confirmed by GC-MS (gas chromatography-mass spectrometry) analysis.

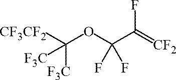

### Example 2: Preparation of 1,1,1,2,2,4,4,4-Octafluoro-3-((Perfluoroallyl)Oxy)-3-(Trifluoromethyl)Butane

Step 1: To a 600 mL stainless steel reaction vessel were added potassium fluoride (17.5 g, 301 mmol) and acetonitrile (125 mL). The reactor was sealed and then evacuated and backfilled with nitrogen three times. After the final evacuation, stirring commenced and perfluoropropionyl fluoride (50.2 g, 302 mmol) was added at a rate which avoided a temperature increase above 30° C. When the reaction temperature fell to 25° C., TMSCF₃ (42.9 g, 302 mmol) was added from a stainless steel cylinder pressurized with argon gas at a rate which did not allow for the reaction mixture temperature to rise above 30° C. The reaction mixture slowly cooled to 25° C. and was allowed to stir at this temperature overnight. The resultant reaction mixture was then transferred to a round-bottom flask and concentrated under reduced pressure, affording potassium 1,1,1,3,3,4,4,4-octafluoro-2-(trifluoromethyl)butan-2-olate (75.4 g, 77% yield) as a white solid. This intermediate was used in the next step without additional purification.

Step 2: To a 3-neck round-bottom flask equipped with a magnetic stir bar, temperature probe, and reflux condenser was charged potassium 1,1,1,3,3,4,4,4-octafluoro-2-(trifluoromethyl)butan-2-olate (15.4 g, 47.5 mmol). The flask was evacuated and backfilled with nitrogen three times followed by the addition of tetraglyme (30 mL). With stirring, the mixture was cooled to 0-5° C. followed by the dropwise addition of perfluoroallyl iodide (11.6 g, 45.1 mmol) at a rate which did not allow for the reaction temperature to rise above 8° C. After complete addition, the mixture was allowed to stir at 0-5° C. for 30 minutes. GC-FID analysis showed approximately 90% conversion of the perfluoroallyl iodide. The reaction was allowed to slowly rise to room temperature and stirred overnight. GC-FID analysis then indicated complete conversion of the perfluoroallyl iodide. The resultant reaction mixture was diluted with water (100 mL) and removal of the aqueous layer afforded a crude fluorochemical mixture which was purified by fractional distillation (108 C, 740 mm/Hg) to give the desired 1,1,1,2,2,4,4,4-octafluoro-3-((perfluoroallyl)oxy)-3-(trifluoromethyl)butane (14.8 g, 55% isolated yield). The identity of the isolated composition was confirmed by GC-MS analysis.

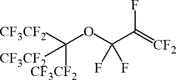

### Example 3: Preparation of 1,1,1,2,2,4,4,5,5,5-Decafluoro-3-((Perfluoroallyl)Oxy)-3-(Perfluoroethyl)Pentane

Step 1: A 600 mL stainless steel pressure reactor was charged with KF (9.6 g, 166 mmol) and acetonitrile (100 mL). The reactor was sealed and then evacuated and backfilled with nitrogen three times. After the final evacuation, stirring commenced and the vessel was charged with perfluoropropionyl fluoride (25.0 g, 151 mmol). After stirring for 10 minutes, TMSCF₂CF₃ (57.9 g, 301 mmol) was slowly added from a stainless steel cylinder pressurized with argon gas at a rate which did not allow for the reaction mixture temperature to rise above 37° C. The reaction mixture slowly cooled to 25 C and was allowed to stir at this temperature overnight. The resultant mixture was concentrated under reduced pressure affording potassium 1,1,1,2,2,4,4,5,5,5-decafluoro-3-(perfluoroethyl)pentan-3-olate as an off-white solid (45.4 g, 71% yield). This intermediate was used in the next step without additional purification.

Step 2: To a 3-neck round-bottom flask equipped with a magnetic stir bar, reflux condenser, and temperature probe was charged 1,1,1,2,2,4,4,5,5,5-decafluoro-3-(perfluoroethyl)pentan-3-olate (20.1 g, 47.4 mmol). The flask was evacuated and backfilled with nitrogen three times followed by the addition of tetraglyme (40 mL). The resultant mixture was cooled to 0° C. with stirring. PFAFS (10.9 g, 47.4 mmol). The reaction mixture was allowed to stir for 30 minutes at a temperature under 10 C. GC-FID analysis indicated complete conversion of PFAFS. Water (50 mL) was then added and the mixture was transferred to a separatory funnel. The aqueous phase was removed affording a fluorochemical layer which was purified via fractional distillation (150-153° C., 740 mm/Hg) affording the desired 1,1,1,2,2,4,4,5,5,5-decafluoro-3-((perfluoroallyl)oxy)-3-(perfluoroethyl)pentane (8.5 g, 35% isolated yield) as a colorless liquid. The identity of the isolated composition was confirmed by GC-MS analysis.

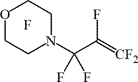

### Example 4 Preparation of 2,2,3,3,5,5,6,6-Octafluoro-4-(Perfluoroallyl)Morpholine

A 3-neck round-bottom flask equipped with a magnetic stir bar, dry ice condenser, and temperature probe was charged with KF (1.5 g, 26 mmol). The reaction vessel was then evacuated and back-filled with nitrogen three times. Tetraglyme (20 mL) was then added to the KF to afford a mixture that was cooled (0° C.) with stirring followed by the slow addition of 2,2,3,3,5,6,6-heptafluoro-1,4-oxazine (5.0 g, 26 mmol). After a 10 minute stir at the same temperature, PFAFS (5.4 g, 23 mmol) was added dropwise at a rate which did not allow for the reaction temperature to increase above 5° C. The resultant reaction mixture was then allowed to slowly warm to room temperature during an overnight stir. GC-FID analysis indicated complete conversion of PFAFS. The contents were transferred to a separatory funnel and the top, organic layer was removed affording the desired 2,2,3,3,5,5,6,6-octafluoro-4-(perfluoroallyl)morpholine (8.1 g, 92% purity, 85% yield determined by GC-FID analysis) as a colorless liquid. The preparation of the desired 2,2,3,3,5,5,6,6-octafluoro-4-(perfluoroallyl)morpholine was confirmed by GC-MS and ¹⁹F NMR (nuclear magnetic reasonance) analyses.

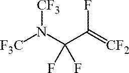

### Example 5. Preparation of 1,1,2,3,3-Pentafluoro-N,N-Bis(Trifluoromethyl)Prop-2-en-1-Amine

A 3-neck round-bottom flask equipped with a magnetic stir bar, temperature probe, and dry ice condenser was charged with KF (2.4 g, 40 mmol). The flask was then evacuated and back-filled with nitrogen three times. Tetraglyme (20 mL) was then added to the KF to afford a mixture that was cooled (0° C.) with stirring followed by the slow addition of 1,1-difluoro-N-(trifluoromethyl)methanimine (5.0 g, 38 mmol). After a 10 minute stir at the same temperature, PFAFS (8.6 g, 38 mmol) was added dropwise at a rate which did not allow for the reaction temperature to rise above 3° C. After complete addition, the reaction mixture was allowed to stir for an additional 1 hour at 0° C. at which point GC-FID analysis indicated >99% conversion of PFAFS. The addition of ice water (50 mL) afforded a mixture that was transferred to a separatory funnel. The aqueous phase was removed to afford the desired 1,1,2,3,3-pentafluoro-N,N-bis(trifluoromethyl)prop-2-en-1-amine (5.2 g, 90% purity, 44% yield determined by GC-FID analysis) as a colorless liquid. The preparation of the desired 2,2,3,3,5,5,6,6-octafluoro-4-(perfluoroallyl)morpholine was confirmed by GC-MS and ¹⁹F NMR analyses.

All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

What is claimed is:

1. A perfluorinated allyl ether compound of formula (I) 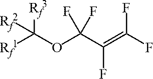
Where R_{f}¹ and R_{f}² are (i) independently selected from a perfluorinated alkyl group comprising 1-7 carbon atoms, a perfluorinated aryl group comprising a 5- or 6-membered ring, or combinations thereof, and optionally comprising one or more catenated heteroatoms selected from N or O; or (ii) bonded together to form a perfluorinated ring structure having 4-8 ring carbon atoms, optionally comprising at least one catenated O or N atom; and
R_{f}³ is a perfluorinated alkyl group comprising 1-2 carbon atoms.

2. The compound of claim 1, wherein R_{f}² is —CF₃, or —CF₂CF₃.

3. The compound of claim 1, wherein R_{f}¹ and R_{f}² are the same.

4. The compound of claim 1, wherein R_{f}¹ and R_{f}² are bonded together to form a four-, five-, or six-membered ring.

5. The compound of claim 1, wherein R_{f}³ is —CF₃, or —CF₂CF₃.

6. The compound of claim 1, wherein R_{f}¹ comprises a perfluorinated pyrrolyl, perfluorinated piperidinyl, or a perfluorinated morpholinyl group.

7. The compound of claim 1, wherein the compound is selected from 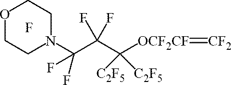 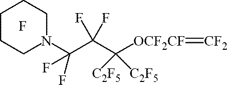 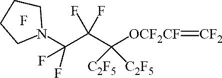 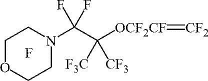 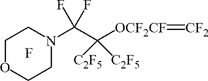 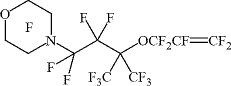 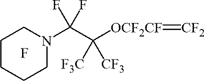 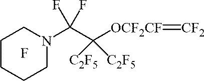 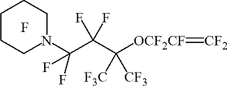 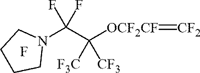 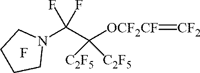 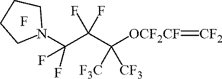 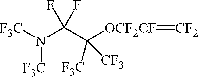 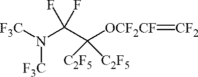 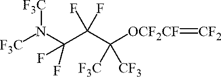 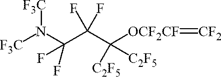 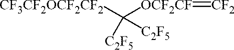 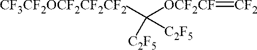 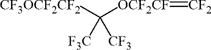 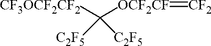 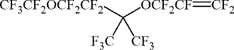 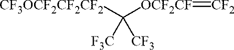 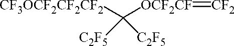 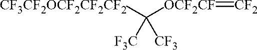 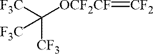 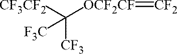 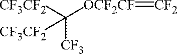 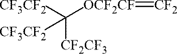 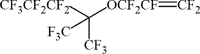 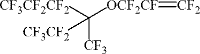 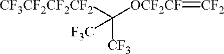 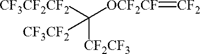 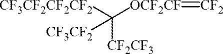 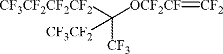 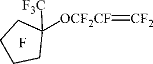 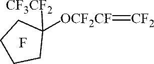 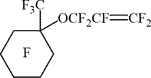 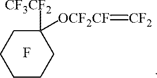
.

8. A method of making a perfluorinated allyl ether comprising:
contacting a perfluoroketone or perfluorinated acid fluoride with a metal or ammonium perfluorocarbanion salt in an aprotic solvent to form a perfluorinated tert-alkoxide salt; and
contacting the perfluorinated tert-alkoxide salt with a perfluoroallylating reagent to form the perfluorinated compound of formula (I) according to claim 1.

9. The method of claim 8, wherein the perfluoroketone and perfluorinated acid fluoride comprise one of the following: 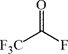 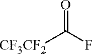 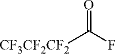 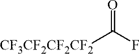 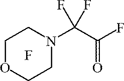 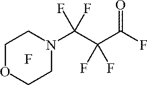 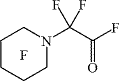 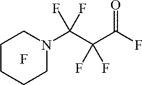 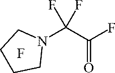 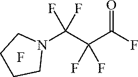 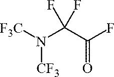 [SMILES: O=C(F)C(F)(F)N(C(F)(F)F)C(F)(F)F] 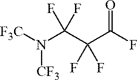 [SMILES: O=C(F)C(F)(F)C(F)(F)N(C(F)(F)F)C(F)(F)F] 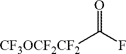 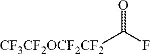 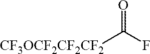 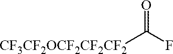 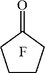 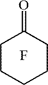 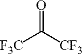
.

10. The method of claim 8, wherein the metal or ammonium perfluorocarbanion salt is generated by contacting tetrafluoroethylene, hexafluoropropylene, (CH₃)₃SiR_{f}³, or (CH₃CH₂)₃SiR_{f}³, where R_{f}³ is a perfluorinated alkyl group comprising 1-2 carbon atoms, with a fluoride salt to generate the perfluorinated tert-alkoxide salt intermediate.

11. A purified compound according to claim 1, wherein the perfluorinated allyl ether compound of formula (I) is at least 90% pure.

12. A polymerizable composition comprising: a fluorinated monomer and the perfluorinated allyl ether compound according to claim 1.

13. The polymerizable composition of claim 12, wherein the fluorinated monomer is at least one of the following: TFE, HFP, VDF, VF, CTFE, a fluorinated vinyl ether, or a fluorinated allyl ether.

14. A method of making a perfluorinated allyl amine comprising:
contacting a perfluorinated imine of Formula (III) with a fluoride salt in an aprotic solvent to form an aza anion salt, where Formula (III) is R_{f}¹—N═CFR_{f}⁴ where (i) R_{f}¹ is a perfluorinated group selected from a perfluorinated alkyl group comprising 1-7 carbon atoms, a perfluorinated aryl group comprising a 5- or 6-membered ring, or combinations thereof, and optionally comprises one or more catenated heteroatoms selected from N or O and R_{f}⁴ is selected from F or a perfluorinated group selected from a perfluorinated alkyl group comprising 1-6 carbon atoms, and a perfluorinated aryl group comprising a 5- or 6-membered ring, or combinations thereof, and optionally comprises one or more catenated heteroatoms selected from N or O; or (ii) R_{f}¹ and R_{f}⁴ are bonded together to form a perfluorinated ring structure having 4-8 ring carbon atoms, optionally comprising at least one catenated O or N atom; and
contacting the aza anion salt with a perfluoroallylating reagent to form a perfluorinated allyl amine of formula (II) 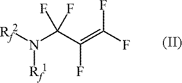
where R_{f}¹ and R_{f}² are (i) independently selected from a perfluorinated alkyl group comprising 1-7 carbon atoms, a perfluorinated aryl group comprising a 5- or 6-membered ring, or a perfluorinated alkaryl ring comprising 1-8 carbon atoms and optionally comprising one or more catenated heteroatoms selected from N or O; or (ii) bonded together to form a perfluorinated ring structure having 4-8 ring carbon atoms, optionally comprising at least one catenated O or N atom.

15. The method of claim 14, wherein the perfluorinated imine of Formula (III) comprise 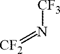 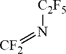 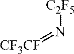 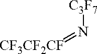 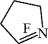 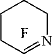 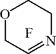 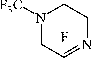 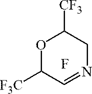 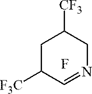 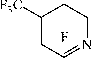 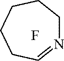
.

16. The method of claim 14, wherein perfluorinated allyl amine of formula (II) is at least 90% pure.

17. The perfluorinated allyl ether compound of claim 1, wherein R_{f}³ is a linear perfluorinated alkyl group.

18. The perfluorinated allyl ether compound of claim 1, wherein R_{f}¹ and R_{f}² are (i) independently selected from a linear perfluorinated alkyl group comprising 1-7 carbon atoms, optionally comprising one or more catenated heteroatoms selected from N or O.
